(19) United States
(12) Patent Application Publication
(10) Pub. No.: US 2024/0324969 A1
(43) Pub. Date: Oct. 3, 2024

(54) DIAGNOSTIC APPARATUS AND DIAGNOSTIC PROGRAM

(71) Applicant: TERUMO KABUSHIKI KAISHA, Tokyo (JP)

(72) Inventor: Takayuki UCHIDA, Hiratsuka-shi (JP)

(73) Assignee: TERUMO KABUSHIKI KAISHA, Tokyo (JP)

(21) Appl. No.: 18/740,727

(22) Filed: Jun. 12, 2024

Related U.S. Application Data
(63) Continuation of application No. PCT/JP2022/045150, filed on Dec. 7, 2022.

(30) Foreign Application Priority Data
Jan. 7, 2022 (JP) 2022-001937

(51) Int. Cl. A61B 5/00; A61B 5/0205; A61B 5/024; A61B 5/11; A61B 5/145; G16H 50/20

(52) U.S. Cl. CPC A61B 5/7275; A61B 5/0205; A61B 5/02405; A61B 5/4041; G16H 50/20; A61B 5/1118; A61B 5/14532

(57) ABSTRACT

A diagnostic apparatus includes a sympathetic nerve activity level index calculation unit that calculates a sympathetic nerve activity level index indicating a sympathetic nerve activity level, a parasympathetic nerve activity level index calculation unit that calculates a parasympathetic nerve activity level index indicating a parasympathetic nerve activity level, and a disease determination unit that determines a possibility of a cardiovascular disease of a subject on the basis of change tendency of the sympathetic nerve activity level index calculated by the sympathetic nerve activity level index calculation unit and change tendency of the parasympathetic nerve activity level index calculated by the parasympathetic nerve activity level index calculation unit.

### CROSS-REFERENCES TO RELATED APPLICATIONS

This application is a continuation of International Application No. PCT/JP2022/045150 filed on Dec. 7, 2022, which claims priority to Japanese Application No. 2022-001937 filed on Jan. 7, 2022, the entire content of both of which is incorporated herein by reference.

### TECHNOLOGICAL FIELD

The present disclosure generally relates to a diagnostic apparatus and a diagnostic program for determining a possibility of a cardiovascular disease such as a stroke.

### BACKGROUND DISCUSSION

In modern times, autonomic nerve disorders have been reported to be associated with various diseases. In addition, utilization of a diagnosis of a state of an autonomic nerve for prediction of risks and prognoses of diseases, detection of onsets of diseases, and the like has been studied. A cardiovascular disease such as a stroke is one disease, which is important to find early after onsets and start treatment. For example, the autonomic nerve is impaired in an acute phase of a stroke, and thus, there is a possibility that an onset of a stroke can be detected at a relatively early stage by monitoring an autonomic nerve function.

Japanese Patent Application Publication No. 2013-46662 A discloses an autonomic nerve function diagnostic apparatus for diagnosing an autonomic nerve function. The autonomic nerve function diagnostic apparatus described in Japanese Patent Application Publication No. 2013-46662 A can independently determine whether a function of a sympathetic nerve and a function of a parasympathetic nerve are normal.

Japanese Patent Application Publication No. 2013-46662 A does not disclose a method for determining a possibility of a cardiovascular disease such as a stroke.

### SUMMARY

A diagnostic apparatus and a diagnostic program are disclosed, which are capable of determining a possibility of a cardiovascular disease of a subject while preventing occurrence of false positive and false negative of a cardiovascular disease such as a stroke.

A diagnostic apparatus according to the present disclosure includes: a sympathetic nerve activity level index calculation unit configured to calculate a sympathetic nerve activity level index indicating a sympathetic nerve activity level; a parasympathetic nerve activity level index calculation unit configured to calculate a parasympathetic nerve activity level index indicating a parasympathetic nerve activity level; and a disease determination unit configured to determine a possibility of a cardiovascular disease of a subject on the basis of change tendency of the sympathetic nerve activity level index calculated by the sympathetic nerve activity level index calculation unit and change tendency of the parasympathetic nerve activity level index calculated by the parasympathetic nerve activity level index calculation unit.

According to the diagnostic apparatus of the present disclosure, the disease determination unit is configured to determine the possibility of the cardiovascular disease of the subject on the basis of the change tendency of the sympathetic nerve activity level index and the change tendency of the parasympathetic nerve activity level index, so that it is possible to prevent occurrence of false positive and false negative of the cardiovascular disease.

In the diagnostic apparatus according to the present disclosure, preferably, the disease determination unit is configured to determine that there is a possibility of the cardiovascular disease in a case where both the sympathetic nerve activity level index calculated by the sympathetic nerve activity level index calculation unit and the parasympathetic nerve activity level index calculated by the parasympathetic nerve activity level index calculation unit tend to decrease.

The sympathetic nerve activity level index and the parasympathetic nerve activity level index tend to change in a contradictory manner at normal times and at times other than an onset of a cardiovascular disease. According to the diagnostic apparatus of the present disclosure, the disease determination unit is configured to determine that there is a possibility of the cardiovascular disease in a case where both the sympathetic nerve activity level index and the parasympathetic nerve activity level index tend to decrease, so that it is possible to further prevent occurrence of false positive and false negative of the cardiovascular disease and determine the possibility of the cardiovascular disease with higher accuracy.

In the diagnostic apparatus according to the present disclosure, preferably, the disease determination unit is configured to determine that there is a possibility of the cardiovascular disease in a case where the sympathetic nerve activity level index calculated by the sympathetic nerve activity level index calculation unit becomes equal to or less than a first ratio with respect to the sympathetic nerve activity level index of the subject when the subject is healthy, and the parasympathetic nerve activity level index calculated by the parasympathetic nerve activity level index calculation unit becomes equal to or less than a second ratio with respect to the parasympathetic nerve activity level index of the subject when the subject is healthy.

According to the diagnostic apparatus of the present disclosure, the disease determination unit is configured to determine that there is a possibility of the cardiovascular disease in a case where the sympathetic nerve activity level index of the subject becomes equal to or less than the first ratio with respect to the sympathetic nerve activity level index of the subject when the subject is healthy and the parasympathetic nerve activity level index of the subject becomes equal to or less than the second ratio with respect to the parasympathetic nerve activity level index of the subject when the subject is healthy, so that it is possible to determine the possibility of the cardiovascular disease with higher accuracy in comparison with the subject when the subject is healthy.

In the diagnostic apparatus according to the present disclosure, preferably, the disease determination unit is configured to determine that there is a possibility of the cardiovascular disease in a case where the sympathetic nerve activity level index calculated by the sympathetic nerve activity level index calculation unit becomes equal to or less than a third ratio with respect to the sympathetic nerve activity level index a predetermined period ago, and the parasympathetic nerve activity level index calculated by the parasympathetic nerve activity level index calculation unit becomes equal to or less than a fourth ratio with respect to the parasympathetic nerve activity level index the predetermined period ago.

According to the diagnostic apparatus of the present disclosure, the disease determination unit is configured to determine that there is a possibility of the cardiovascular disease in a case where the sympathetic nerve activity level index of the subject becomes equal to or less than the third ratio with respect to the sympathetic nerve activity level index of the subject himself/herself the predetermined period ago and the parasympathetic nerve activity level index of the subject becomes equal to or less than the fourth ratio with respect to the parasympathetic nerve activity level index of the subject himself/herself the predetermined period ago. It is therefore possible to prevent occurrence of false positive and false negative of the cardiovascular disease due to influence of diurnal variability of the sympathetic nerve activity level index and the parasympathetic nerve activity level index and to determine the possibility of the cardiovascular disease with higher accuracy.

In the diagnostic apparatus according to the present disclosure, preferably, the disease determination unit is configured to determine that there is a possibility of the cardiovascular disease in a case where the sympathetic nerve activity level index calculated by the sympathetic nerve activity level index calculation unit becomes equal to or less than a first predetermined value smaller than the sympathetic nerve activity level index a predetermined period ago, and the parasympathetic nerve activity level index calculated by the parasympathetic nerve activity level index calculation unit becomes equal to or less than a second predetermined value smaller than the parasympathetic nerve activity level index the predetermined period ago.

According to the diagnostic apparatus of the present disclosure, the disease determination unit is configured to determine that there is a possibility of the cardiovascular disease in a case where the sympathetic nerve activity level index of the subject becomes equal to or less than the first predetermined value smaller than the sympathetic nerve activity level index of the subject himself/herself the predetermined period ago and the parasympathetic nerve activity level index of the subject becomes equal to or less than the second predetermined value smaller than the parasympathetic nerve activity level index of the subject himself/herself the predetermined period ago. It is therefore possible to prevent occurrence of false positive and false negative of the cardiovascular disease due to influence of diurnal variability of the sympathetic nerve activity level index and the parasympathetic nerve activity level index and to determine the possibility of the cardiovascular disease with higher accuracy.

In the diagnostic apparatus according to the present disclosure, preferably, the first predetermined value is determined on the basis of a change in the sympathetic nerve activity level index the predetermined period ago and a change in the sympathetic nerve activity level index in the same hours on or before the previous day, and/or the second predetermined value is determined on the basis of a change in the parasympathetic nerve activity level index the predetermined period ago and a change in the parasympathetic nerve activity level index in the same hours on or before the previous day.

According to the diagnostic apparatus of the present disclosure, the first predetermined value is determined on the basis of the change in the sympathetic nerve activity level index the predetermined period ago and the change in the sympathetic nerve activity level index in the same hours on or before the previous day. The second predetermined value is determined on the basis of the change in the parasympathetic nerve activity level index the predetermined period ago and the change in the parasympathetic nerve activity level index of the same hours on or before the previous day. This makes it possible to further prevent occurrence of false positive and false negative of the cardiovascular disease due to influence of diurnal variability of the sympathetic nerve activity level index and the parasympathetic nerve activity level index, and to determine the possibility of the cardiovascular disease with higher accuracy.

In the diagnostic apparatus according to the present disclosure, preferably, the cardiovascular disease is a stroke.

According to the diagnostic apparatus of the present disclosure, it is possible to prevent occurrence of false positive and false negative of the stroke among cardiovascular diseases.

In the diagnostic apparatus according to the present disclosure, preferably, the disease determination unit is configured to determine that there is a possibility of the cardiovascular disease in a case where the sympathetic nerve activity level index calculated by the sympathetic nerve activity level index calculation unit becomes equal to or less than a fifth ratio with respect to the sympathetic nerve activity level index in the same hours on or before the previous day, and the parasympathetic nerve activity level index calculated by the parasympathetic nerve activity level index calculation unit becomes equal to or less than a sixth ratio with respect to the parasympathetic nerve activity level index in the same hours.

According to the diagnostic apparatus of the present disclosure, the disease determination unit is configured to determine that there is a possibility of the cardiovascular disease in a case where the sympathetic nerve activity level index of the subject becomes equal to or less than the fifth ratio with respect to the sympathetic nerve activity level index of the subject himself/herself in the same hours on or before the previous day and the parasympathetic nerve activity level index of the subject becomes equal to or less than the sixth ratio with respect to the parasympathetic nerve activity level index of the subject himself/herself in the same hours on or before the previous day. It is therefore possible to prevent occurrence of false positive and false negative of the cardiovascular disease due to influence of diurnal variability within a predetermined period of the sympathetic nerve activity level index and the parasympathetic nerve activity level index and to determine the possibility of the cardiovascular disease with higher accuracy.

Preferably, the diagnostic apparatus according to the present disclosure further includes a heartbeat interval calculation unit configured to calculate a heartbeat interval of the subject, in which the sympathetic nerve activity level index calculation unit is configured to calculate the sympathetic nerve activity level index on the basis of heart rate variability indicating variability of the heartbeat interval calculated by the heartbeat interval calculation unit, and the parasympathetic nerve activity level index calculation unit is configured to calculate the parasympathetic nerve activity level index on the basis of the heart rate variability.

According to the diagnostic apparatus of the present disclosure, the sympathetic nerve activity level index calculation unit is configured to calculate the sympathetic nerve activity level index on the basis of the heart rate variability to be used as an autonomic nerve index. The parasympathetic nerve activity level index calculation unit is configured to calculate the parasympathetic nerve activity level index on the basis of the heart rate variability to be used as the autonomic nerve index. For example, in a case where the heartbeat interval calculation unit is configured to calculate the heartbeat interval on the basis of an electrocardiogram and invasive blood pressure data, the disease determination unit can determine the possibility of the cardiovascular disease with higher accuracy. For example, in a case where the heartbeat interval calculation unit is configured to calculate the heartbeat interval on the basis of data measured by a wearable terminal, or the like, it is possible to prevent a scale of the diagnostic apparatus from becoming large and to reduce a burden associated with measurement to be felt by the subject, and the disease determination unit can easily determine the possibility of the cardiovascular disease even in a state where the subject is living his/her daily life.

In the diagnostic apparatus according to the present disclosure, preferably, the disease determination unit is configured to determine the possibility of the cardiovascular disease further on the basis of change tendency of the heartbeat interval calculated by the heartbeat interval calculation unit.

According to the diagnostic apparatus of the present disclosure, the disease determination unit is configured to determine the possibility of the cardiovascular disease on the basis of the change tendency (time domain analysis, or the like) of the heartbeat interval calculated by the heartbeat interval calculation unit, so that it is possible to determine the possibility of the cardiovascular disease with higher accuracy.

In the diagnostic apparatus according to the present disclosure, preferably, the sympathetic nerve activity level index calculation unit is configured to calculate the sympathetic nerve activity level index on the basis of frequency component analysis of the heart rate variability, and the parasympathetic nerve activity level index calculation unit is configured to calculate the parasympathetic nerve activity level index on the basis of the frequency component analysis of the heart rate variability.

According to the diagnostic apparatus of the present disclosure, the sympathetic nerve activity level index calculation unit is configured to calculate the sympathetic nerve activity level index by analyzing a frequency component of the heart rate variability using, for example, a frequency component analysis method such as fast Fourier transform. In addition, the parasympathetic nerve activity level index calculation unit is configured to calculate the parasympathetic nerve activity level index by analyzing a frequency component of the heart rate variability using, for example, a frequency component analysis method such as fast Fourier transform. Thus, the disease determination unit can determine the possibility of the cardiovascular disease with higher accuracy.

The diagnostic apparatus according to the present disclosure preferably further includes a biological information measurement unit configured to measure biological information including at least any one of a blood pressure, a body temperature, or a blood gas of the subject, in which the disease determination unit is configured to determine the possibility of the cardiovascular disease further on the basis of change tendency of the biological information measured by the biological information measurement unit.

The blood pressure, the body temperature and the blood gas tend to change immediately after an onset of the cardiovascular disease. According to the diagnostic apparatus of the present disclosure, the disease determination unit is configured to determine the possibility of the cardiovascular disease of the subject on the basis of not only the change tendency of the sympathetic nerve activity level index and the parasympathetic nerve activity level index but also the change tendency of at least any one of the blood pressure, the body temperature, or the blood gas of the subject, so that it is possible to further prevent occurrence of false positive and false negative of the cardiovascular disease and determine the possibility of the cardiovascular disease with higher accuracy.

The diagnostic apparatus according to the present disclosure preferably further includes a subject information input unit to which subject information including at least any one of information regarding daily movement of the subject, information regarding a drug to be used by the subject, or information regarding a basic disease of the subject is input, in which the disease determination unit is configured to determine the possibility of the cardiovascular disease further on the basis of the subject information input to the subject information input unit.

The sympathetic nerve activity level index and the parasympathetic nerve activity level index may also change depending on daily movement such as standing, meal, and sleeping of the subject, administration of a drug to the subject, medication to the subject, or a basic disease of the subject. According to the diagnostic apparatus of the present disclosure, the disease determination unit is configured to determine the possibility of the cardiovascular disease of the subject on the basis of the change tendency of the sympathetic nerve activity level index and the parasympathetic nerve activity level index in consideration of at least any one of the information regarding the daily movement of the subject, the information regarding the drug to be used by the subject, or the information regarding the basic disease of the subject. It is therefore possible to prevent occurrence of false positive and false negative of the cardiovascular disease due to influence of the daily movement, administration of a drug/medication, and the basic disease of the subject and to determine the possibility of the cardiovascular disease with higher accuracy.

Preferably, the diagnostic apparatus according to the present disclosure further includes a body motion measurement unit configured to measure body motion of the subject, and the disease determination unit is configured to determine the possibility of the cardiovascular disease further on the basis of the body motion measured by the body motion measurement unit.

The sympathetic nerve activity level index and the parasympathetic nerve activity level index may also change depending on the body motion of the subject. According to the diagnostic apparatus of the present disclosure, the disease determination unit is configured to determine the possibility of the cardiovascular disease of the subject on the basis of the change tendency of the sympathetic nerve activity level index and the parasympathetic nerve activity level index in consideration of the body motion of the subject, so that it is possible to prevent occurrence of false positive and false negative of the cardiovascular disease due to influence of the body motion of the subject and to determine the possibility of the cardiovascular disease with higher accuracy.

Preferably, the diagnostic apparatus according to the present disclosure further includes a blood glucose measurement unit configured to measure blood glucose of the subject, and the disease determination unit is configured to determine the possibility of the cardiovascular disease further on the basis of change tendency of the blood glucose measured by the blood glucose measurement unit.

The sympathetic nerve activity level index and the parasympathetic nerve activity level index may also change depending on a change in the blood glucose of the subject. According to the diagnostic apparatus of the present disclosure, the disease determination unit determines the possibility of the cardiovascular disease of the subject on the basis of the change tendency of the sympathetic nerve activity level index and the parasympathetic nerve activity level index in consideration of the blood glucose tendency of the subject, so that it is possible to prevent occurrence of false positive and false negative of the cardiovascular disease due to influence of the blood glucose of the subject and to determine the possibility of the cardiovascular disease with higher accuracy.

A non-transitory computer-readable medium storing a diagnostic program according to the present disclosure that causes a computer to execute: a sympathetic nerve activity level index calculation procedure of calculating a sympathetic nerve activity level index indicating a sympathetic nerve activity level; a parasympathetic nerve activity level index calculation procedure of calculating a parasympathetic nerve activity level index indicating a parasympathetic nerve activity level; and a disease determination procedure of determining a possibility of a cardiovascular disease of a subject on the basis of change tendency of the sympathetic nerve activity level index calculated by the sympathetic nerve activity level index calculation procedure and change tendency of the parasympathetic nerve activity level index calculated by the parasympathetic nerve activity level index calculation procedure.

According to the diagnostic program of the present disclosure, the diagnostic program causes the computer to execute the disease determination procedure for determining the possibility of the cardiovascular disease of the subject on the basis of the change tendency of the sympathetic nerve activity level index and the change tendency of the parasympathetic nerve activity level index, so that it is possible to prevent occurrence of false positive and false negative of the cardiovascular disease.

A method for determining a possibility of a cardiovascular disease of a subject according to the present disclosure includes calculating a sympathetic nerve activity level index indicating a sympathetic nerve activity level; calculating a parasympathetic nerve activity level index indicating a parasympathetic nerve activity level; and determining the possibility of the cardiovascular disease of the subject on a basis of change tendency of the sympathetic nerve activity level index calculated by the sympathetic nerve activity level index calculation unit and change tendency of the parasympathetic nerve activity level index calculated by the parasympathetic nerve activity level index calculation unit.

According to the present disclosure, it is possible to provide a diagnostic apparatus and a diagnostic program capable of determining a possibility of a cardiovascular disease of a subject while preventing occurrence of false positive and false negative of the cardiovascular disease such as a stroke.

### BRIEF DESCRIPTION OF THE DRAWINGS

FIG. 1 is a block diagram illustrating a diagnostic apparatus according to an embodiment of the present disclosure.

FIG. 2 is a table indicating a first specific example of determination made by a disease determination unit of the present embodiment.

FIG. 3 is a table indicating a second specific example of determination made by the disease determination unit of the present embodiment.

FIG. 4 is a table indicating a third specific example of determination made by the disease determination unit of the present embodiment.

FIG. 5 is a table indicating a time domain index and a frequency domain index of heart rate variability.

FIG. 6 is a graph indicating change tendency of a sympathetic nerve activity level index (LF/HF) at an onset of a stroke.

FIG. 7 is a graph indicating change tendency of a parasympathetic nerve activity level index (HF) at an onset of a stroke.

FIG. 8 is a graph indicating change tendency of a sympathetic nerve activity level index (LF/HF) at the time of a change in body position to a standing position which is one of body positions.

FIG. 9 is a graph indicating change tendency of a parasympathetic nerve activity level index (HF) at the time of a change in body position to a standing position which is one of body positions.

FIG. 10 is a graph for explaining a case where the disease determination unit of the present embodiment determines a possibility of a stroke further on the basis of a blood pressure as additional information.

FIG. 11 is a table for explaining a case where the disease determination unit of the present embodiment determines the possibility of the stroke further on the basis of a blood gas as the additional information.

### DETAILED DESCRIPTION

Set forth below with reference to the accompanying drawings is a detailed description of embodiments of a diagnostic apparatus and a diagnostic program for determining a possibility of a cardiovascular disease such as a stroke.

Note that the embodiment described below is a preferred specific example of the present disclosure, and thus various technically preferable limitations are given. However, the scope of the present disclosure is not limited to these aspects unless there is a description to limit the present disclosure in the following description. Furthermore, in the drawings, the same components are denoted by the same reference signs, and the detailed description of the same components are omitted.

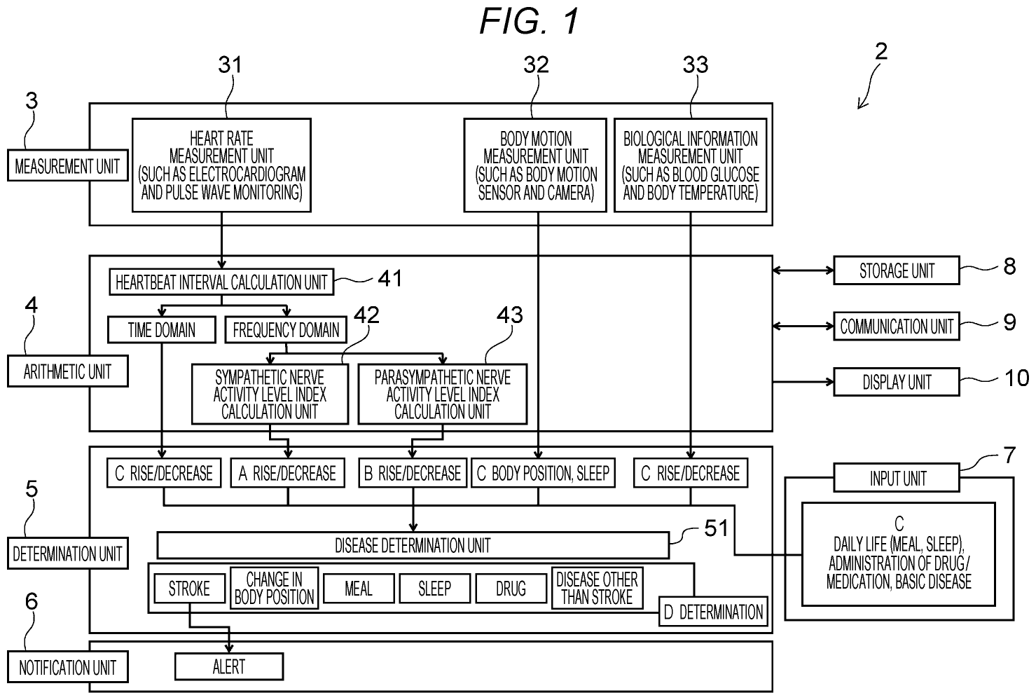

FIG. 1 is a block diagram illustrating a diagnostic apparatus according to an embodiment of the present disclosure.

A diagnostic apparatus 2 according to the present embodiment is an apparatus that determines a possibility of a cardiovascular disease such as a stroke, and can include an arithmetic unit 4, a determination unit 5, a storage unit 8, and a communication unit 9. The diagnostic apparatus 2 may further include a measurement unit 3, a notification unit 6, an input unit 7, and a display unit 10. In accordance with an embodiment, the measurement unit 3, the notification unit 6, the input unit 7, and the display unit 10 may be provided as part of the diagnostic apparatus 2 or may be provided as another apparatus different from the diagnostic apparatus 2.

The cardiovascular disease can include, but are not limited to, a stroke (cerebral infarction, cerebral hemorrhage, subarachnoid hemorrhage, etc.), and a heart disease (ischemic heart disease such as acute myocardial infarction, heart failure, atrial fibrillation, angina pectoris, etc.).

The measurement unit 3 can include a heart rate measurement unit 31, a body motion measurement unit 32, and a biological information measurement unit 33.

The heart rate measurement unit 31 measures heartbeat, that is, a heart rate of the subject. Examples of the heart rate measurement unit 31 can include an electrocardiogram monitor, a pulse wave monitor, and a sphygmomanometer. The heart rate measurement unit 31 is not limited to an apparatus to be used in a hospital, and a wristband-type wearable terminal, or the like, which is used outside the hospital and can measure the heart rate is preferable because it allows monitoring of a cardiovascular disease even in a state where the subject is living his/her daily life.

The body motion measurement unit 32 measures body motion or a body position of the subject. The body motion referred to herein is a change in posture, that is, motion associated with a change in posture such as from standing to sitting, from standing to lying, from sitting to lying, from sitting to standing, from lying to sitting, from lying to standing, from lying to sitting, from lying to standing, and the like, minute motion in association with breathing during sleep, and the like, and does not include motion of the body due to daily movement, sports, or walking. Examples of the body motion measurement unit 32 include a body motion sensor installed under a bed, a camera, an ultrasonic measurement device, a three-axis acceleration sensor attached to a subject, and the like.

Examples of the body motion sensor include a sensor that detects body motion in association with breathing or rolling over by detecting a pressure change by a pressure sensor built in an air mat. In addition, a wearable measurement device including a three-axis acceleration sensor can be applied as the body motion sensor.

The biological information measurement unit 33 measures biological information including a blood pressure, a body temperature, and a blood gas of the subject. In addition, the biological information measurement unit 33 may measure blood glucose of the subject. Thus, a “blood glucose measurement unit” of the present disclosure may be included in the biological information measurement unit 33. Examples of the biological information measurement unit 33 include thermography, a blood glucose meter, and the like.

The arithmetic unit 4 acquires various kinds of information from the measurement unit 3 and the input unit 7, reads a program stored in the storage unit 8 and executes various kinds of calculation and processing. Alternatively, the arithmetic unit 4 acquires various kinds of information from another apparatus via the communication unit 9, reads a program stored in the storage unit 8 and executes various kinds of calculation and processing. Examples of the arithmetic unit 4 can include a central processing unit (CPU) and a field programmable gate array (FPGA).

The arithmetic unit 4 can include a heartbeat interval calculation unit 41, a sympathetic nerve activity level index calculation unit 42, and a parasympathetic nerve activity level index calculation unit 43.

The heartbeat interval calculation unit 41 calculates a heartbeat interval of the subject on the basis of data measured by the heart rate measurement unit 31. For example, the heartbeat interval calculation unit 41 calculates an interval between R waves of an electrocardiogram, that is, an interval (R-R interval) between vertices of R waves adjacent to each other in a waveform of the electrocardiogram as the heartbeat interval on the basis of the electrocardiogram measured by the electrocardiogram monitoring. In addition, the heartbeat interval calculation unit 41 calculates variability of the heartbeat interval (heart rate variability) of the subject. For example, the heartbeat interval calculation unit 41 calculates variability in the interval of the R waves of the electrocardiogram, that is, variability in an instantaneous heart rate as the heart rate variability.

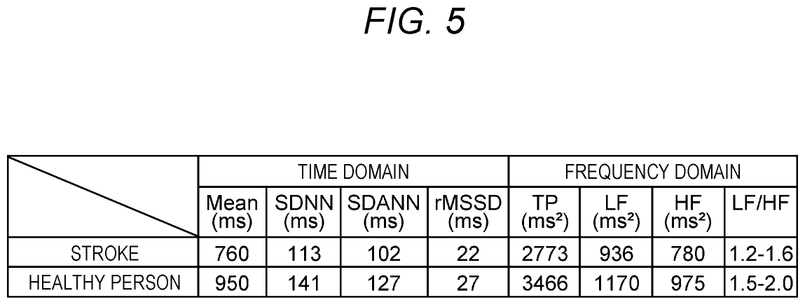

Furthermore, the heartbeat interval calculation unit 41 executes time domain analysis of the heart rate variability, and calculates, as a time domain index of the heart rate variability, a mean value (Mean) of the R-R intervals (RRI), standard deviation (SDNN) of the R-R intervals, standard deviation (SDANN) over 24 hours of the average R-R interval for each of 5 minutes, and a square root of a mean of squares of a difference between the adjacent R-R intervals (rMSSD) (see FIG. 5). In addition, the heartbeat interval calculation unit 41 executes frequency component analysis (for example, fast Fourier transform (FFT)) of the heart rate variability, and calculates a low frequency component LF, a high frequency component HF, a ratio LF/HF of the low frequency component (LF)/high frequency component (HF), and a power value TP of the entire frequency band as a frequency domain index of the heart rate variability (see FIG. 5). For example, a component of 0.15 Hz or less of the heart rate variability can be set as the low frequency component, and a component of 0.15 Hz or more of the heart rate variability can be set as the high frequency component. However, an upper limit of the low frequency component and a lower limit of the high frequency component are not limited to this frequency. For frequency bands for calculating the components, it is only necessary that the high frequency component is in a frequency band relatively higher than a frequency band for the low frequency component. In addition, the frequency band for calculating the low frequency component and the frequency band for calculating the high frequency component may partially overlap each other.

The analysis may be performed on the accumulated data at timings for each of a predetermined number of days (for example, for each day) or timings in real time or for each of a short period (for example, five minutes). Furthermore, in addition to the above items, the heartbeat interval calculation unit 41 may calculate other items related to the heart rate variability, such as a coefficient of variation of RRI (CVRR, value obtained by dividing SDNN by a mean value of RRI), an NN50 (total number of RRI differences exceeding 50 ms which are consecutively adjacent), a PNN50 (ratio of heart rates for which difference in the RRI which are consecutively adjacent exceeds 50 ms), and a VLF (ultra-low frequency band).

Note that the heartbeat interval calculation unit 41 may calculate the heartbeat interval and the heart rate variability on the basis of measurement data other than the electrocardiogram. For example, the heartbeat interval calculation unit 41 may calculate the heartbeat interval and the heart rate variability on the basis of a pulse wave and an invasive blood pressure of the subject. Thus, as described above, the heart rate measurement unit 31 is not limited to the electrocardiogram monitoring and may be pulse wave monitoring, a sphygmomanometer, or the like. In the present embodiment, a case where the heart rate measurement unit 31 performs electrocardiogram monitoring will be described as an example.

The sympathetic nerve activity level index calculation unit 42 calculates a sympathetic nerve activity level index indicating a sympathetic nerve activity level on the basis of the heart rate variability calculated by the heartbeat interval calculation unit 41. For example, the sympathetic nerve activity level index calculation unit 42 calculates LF/HF as the sympathetic nerve activity level index on the basis of the low frequency component LF and the high frequency component HF calculated by the heartbeat interval calculation unit 41 (see FIG. 5). “LF/HF” is one of the frequency domains of the sympathetic nerve activity level index.

The parasympathetic nerve activity level index calculation unit 43 calculates a parasympathetic nerve activity level index indicating a parasympathetic nerve activity level on the basis of the heart rate variability calculated by the heartbeat interval calculation unit 41. For example, the parasympathetic nerve activity level index calculation unit 43 sets the high frequency component HF calculated by the heartbeat interval calculation unit 41 as the parasympathetic nerve activity level index (see FIG. 5). “HF” is one of the frequency domains of the parasympathetic nerve activity level index.

The determination unit 5 includes a disease determination unit 51. Examples of the determination unit 5 include a CPU and an FPGA. The disease determination unit 51 determines a possibility of a cardiovascular disease of the subject on the basis of change tendency (A: rise/decrease) of the sympathetic nerve activity level index (LF/HF) calculated by the sympathetic nerve activity level index calculation unit 42 and change tendency (B: rise/decrease) of the parasympathetic nerve activity level index (HF) calculated by the parasympathetic nerve activity level index calculation unit 43. This will be described later in detail.

In addition, the disease determination unit 51 may determine the possibility of the cardiovascular disease of the subject further on the basis of change tendency of the heartbeat interval calculated by the heartbeat interval calculation unit 41. In other words, the disease determination unit 51 may determine the possibility of the cardiovascular disease of the subject further on the basis of change tendency (C: rise/decrease) of at least any one of Mean, SDNN, SDANN, or rMSSD calculated as a time domain index of the heart rate variability by the heartbeat interval calculation unit 41.

Furthermore, the disease determination unit 51 may determine the possibility of the cardiovascular disease of the subject further on the basis of the body motion or posture (C: body motion associated with change in posture such as standing to sitting, standing to lying, sitting to lying, sitting to standing, lying to sitting, and lying to standing, and minute body motion in association with breathing during sleep) of the subject measured by the body motion measurement unit 32. In addition, the disease determination unit 51 may determine the possibility of the cardiovascular disease of the subject further on the basis of change tendency (C: rise/decrease) in biological information (a blood pressure, a body temperature, a blood gas, blood glucose, etc.) of the subject measured by the biological information measurement unit 33.

The input unit 7 receives input of subject information including at least any one of information regarding daily movement (meal, sleep, etc.) of the subject, information regarding a drug to be used by the subject, or information regarding a basic disease of the subject. The input unit 7 of the present embodiment is an example of a “subject information input unit” of the present disclosure. The subject information may be input using a method in which the subject information is input by the subject himself/herself or a method in which the subject information is automatically input from the biological information measurement device. The input unit 7 transmits the input subject information to the determination unit 5. The input unit 7 may transmit the subject information to the determination unit 5 via the communication unit 9. Examples of the input unit 7 include a portable electronic terminal apparatus such as a tablet computer. As for the meal, a blood glucose level of the subject may be monitored by a blood glucose meter, and it may be determined that the subject has had a meal when the blood glucose level rises.

The disease determination unit 51 may determine the possibility of the cardiovascular disease further on the basis of the subject information (C: daily movement, administration of a drug/medication, a basic disease) input to the input unit 7.

The notification unit 6 gives a notification of an alert in a case where the disease determination unit 51 determines that the subject has a possibility of the cardiovascular disease. For example, the notification unit 6 gives a notification that the subject has a possibility of the cardiovascular disease by sound, light, or the like. Alternatively, the notification unit 6 may display on the display unit 10 that the subject has a possibility of the cardiovascular disease. The notification unit 6 may notify not only the subject but also a family member of the subject or a medical institution near the home of the subject or used by the subject of the alert by communication.

The storage unit 8 stores various programs such as a calculation program for controlling calculation operation of the heartbeat interval calculation unit 41, the sympathetic nerve activity level index calculation unit 42, and the parasympathetic nerve activity level index calculation unit 43, a determination program for controlling determination operation of the disease determination unit 51, a notification program for controlling notification operation of the notification unit 6, and a display program for controlling display operation of the display unit 10. In addition, the storage unit 8 stores various kinds of data such as calculation data of the heartbeat interval calculation unit 41, the sympathetic nerve activity level index calculation unit 42, and the parasympathetic nerve activity level index calculation unit 43, and determination data of the disease determination unit 51. Note that the program is not limited to being stored in the storage unit 8 and the program may be stored in advance in a computer-readable storage medium and distributed, or may be downloaded to the diagnostic apparatus 2 via a network.

Examples of the storage unit 8 include a semiconductor memory built in the diagnostic apparatus 2. Alternatively, examples of the storage unit 8 include various storage media such as a compact disc (CD), a digital versatile disc (DVD), a random access memory (RAM), a read only memory (ROM), a hard disk, and a memory card connectable to the diagnostic apparatus 2, a data server, and the like.

The heartbeat interval calculation unit 41, the sympathetic nerve activity level index calculation unit 42, and the parasympathetic nerve activity level index calculation unit 43 are implemented by the arithmetic unit 4 executing a program stored (stored) in the storage unit 8. The disease determination unit 51 is implemented by the arithmetic unit 4 executing a program stored (stored) in the storage unit 8. Note that the heartbeat interval calculation unit 41, the sympathetic nerve activity level index calculation unit 42, the parasympathetic nerve activity level index calculation unit 43, and the disease determination unit 51 may be implemented by hardware or may be implemented by a combination of hardware and software.

The program to be executed by the computer including the arithmetic unit 4 and the determination unit 5 corresponds to a “diagnostic program” of the present disclosure. The “computer” as used herein is not limited to a personal computer and includes an arithmetic processing apparatus and a microcomputer which are included in an information processing apparatus, and collectively refers to a device and an apparatus which are capable of implementing the functions of the present disclosure with a program.

Here, the autonomic nerve includes a sympathetic nerve that mainly functions in an active state and a parasympathetic nerve that mainly functions in a resting state. When the living body is in a tension/active state, a state of the autonomic nerve becomes a sympathetic nerve dominant state. On the other hand, when the living body is in a resting state, the state of the autonomic nerve becomes a parasympathetic nerve dominant state. As described above, the activity levels of the sympathetic nerve and the parasympathetic nerve are in a contradictory relationship and vary within a day. For example, the sympathetic nerve activity is dominant in the daytime, whereas the parasympathetic nerve is dominant at night. Furthermore, the activity levels of the sympathetic nerve and the parasympathetic nerve may vary in a short period of a day in association with body motion of the subject caused by a change in posture such as from standing to sitting, from standing to lying, from sitting to lying, from sitting to standing, from lying to sitting, from lying to standing. In addition, even in a case where movement of the subject is relatively small, the activity levels of the sympathetic nerve and the parasympathetic nerve may vary depending on daily movement, meal, sleep, administration of a drug, medication, and the like. Furthermore, the activity levels of the sympathetic nerve and the parasympathetic nerve may vary in a case of diseases other than the cardiovascular disease such as a stroke. Thus, if a possibility of the cardiovascular disease such as a stroke is determined only by setting a threshold regarding the activity levels of the sympathetic nerve and the parasympathetic nerve and determining abnormality of the sympathetic nerve and the parasympathetic nerve, false positive and false negative of the cardiovascular disease may occur.

On the other hand, the disease determination unit 51 of the diagnostic apparatus 2 according to the present embodiment determines the possibility of the cardiovascular disease of the subject on the basis of the change tendency of the sympathetic nerve activity level index calculated by the sympathetic nerve activity level index calculation unit 42 and the change tendency of the parasympathetic nerve activity level index calculated by the parasympathetic nerve activity level index calculation unit 43. More specifically, according to the findings obtained by the present inventors, in a case where there is a possibility of an onset of a stroke, both the sympathetic nerve activity level index and the parasympathetic nerve activity level index tend to decrease. Thus, the disease determination unit 51 of the diagnostic apparatus 2 according to the present embodiment determines that there is a possibility of an onset of a stroke in a case where both the sympathetic nerve activity level index calculated by the sympathetic nerve activity level index calculation unit 42 and the parasympathetic nerve activity level index calculated by the parasympathetic nerve activity level index calculation unit 43 tend to decrease.

According to the diagnostic apparatus 2 of the present embodiment, the disease determination unit 51 determines the possibility of the cardiovascular disease of the subject on the basis of the change tendency of the sympathetic nerve activity level index calculated by the sympathetic nerve activity level index calculation unit 42 and the change tendency of the parasympathetic nerve activity level index calculated by the parasympathetic nerve activity level index calculation unit 43. It is therefore possible to prevent occurrence of false positive and false negative of the cardiovascular disease. In addition, on the basis of the findings obtained by the present inventors that the sympathetic nerve activity level index and the parasympathetic nerve activity level index tend to change in a contradictory manner at normal times and at times other than an onset of a stroke, the disease determination unit 51 determines that there is a possibility of an onset of a stroke in a case where both the sympathetic nerve activity level index calculated by the sympathetic nerve activity level index calculation unit 42 and the parasympathetic nerve activity level index calculated by the parasympathetic nerve activity level index calculation unit 43 tend to decrease. Thus, the disease determination unit 51 can further prevent occurrence of false positive and false negative of the cardiovascular disease and determine a possibility of an acute cardiovascular disease with higher accuracy.

Hereinafter, the determination operation of the disease determination unit 51 will be further described with reference to the drawings.

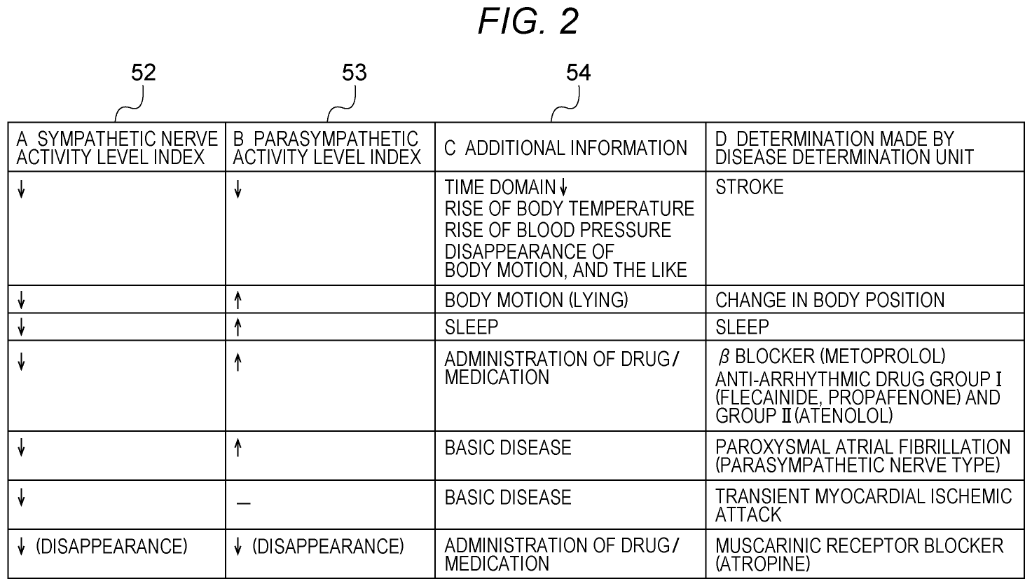

FIG. 2 is a table indicating a first specific example of determination made by the disease determination unit of the present embodiment.

As indicated in FIG. 2, in the first specific example of the determination made by the disease determination unit 51, a case where the sympathetic nerve activity level index 52 tends to decrease will be described.

In the following description, the sympathetic nerve activity level index calculated by the sympathetic nerve activity level index calculation unit 42 may be simply referred to as a “sympathetic nerve activity level index 52”. The parasympathetic nerve activity level index calculated by the parasympathetic nerve activity level index calculation unit 43 may be simply referred to as a “parasympathetic nerve activity level index 53”. The subject information including information regarding daily movement (meal, sleep, etc.) of the subject, information regarding a drug to be used by the subject, and information regarding a basic disease of the subject input to the input unit 7, the time domain index of the heart rate variability calculated by the heartbeat interval calculation unit 41, the body motion (a body position, sleep) of the subject measured by the body motion measurement unit 32, and the biological information (a blood pressure, a body temperature, a blood gas, blood glucose, etc.) of the subject measured by the biological information measurement unit 33 may be simply referred to as “additional information 54”.

In the following description, a case will be described as an example where the disease determination unit 51 determines a possibility of a “stroke” as an example of the cardiovascular disease. A case will be described as an example where the sympathetic nerve activity level index calculation unit 42 calculates LF/HF as the sympathetic nerve activity level index 52 on the basis of the low frequency component LF and the high frequency component HF calculated by the heartbeat interval calculation unit 41. A case will be described as an example where the parasympathetic nerve activity level index calculation unit 43 sets the high frequency component HF calculated by the heartbeat interval calculation unit 41 as the parasympathetic nerve activity level index.

As indicated in FIG. 2, for example, if there is a change in body position (body motion) to a lying position, sleep, administration/medication of a specific drug, and an onset of a specific acute disease, the sympathetic nerve activity level index 52 may tend to decrease, while the parasympathetic nerve activity level index 53 may tend to increase. In this case, the disease determination unit 51 determines that there is no possibility of a stroke, and there is a possibility of a change in body position to a lying position, sleep, or the like, as indicated in FIG. 2.

In addition, as indicated in FIG. 2, for example, if a specific acute disease occurs, the parasympathetic nerve activity level index 53 may hardly change while the sympathetic nerve activity level index 52 tends to decrease. In this case, the disease determination unit 51 determines that there is no possibility of a stroke, and there is a possibility of a transient myocardial ischemic attack, for example, together with the additional information of the basic disease as indicated in FIG. 2. Furthermore, as indicated in FIG. 2, for example, if there is administration/medication of a muscarinic receptor blocker, both the sympathetic nerve activity level index 52 and the parasympathetic nerve activity level index 53 tend to decrease, but tend to decrease to a level of disappearance (i.e., ceases to exist). In this case, the disease determination unit 51 determines that there is no possibility of a stroke and that there is a possibility that administration/medication of a muscarinic receptor blocker (atropine) have been performed as indicated in FIG. 2.

On the other hand, as indicated in FIG. 2, in a case where both the sympathetic nerve activity level index 52 and the parasympathetic nerve activity level index 53 tend to decrease, the disease determination unit 51 determines that the subject has a possibility of a stroke.

According to this, the disease determination unit 51 can further prevent occurrence of false positive and false negative of the stroke and determine the possibility of the stroke with higher accuracy. In addition, in a case where the possibility of the stroke of the subject is determined further on the basis of the time domain index of the heart rate variability calculated by the heartbeat interval calculation unit 41 as the additional information 54, the disease determination unit 51 can determine the possibility of the stroke with higher accuracy. Furthermore, in a case where the possibility of the stroke of the subject is determined further on the basis of the body motion (change in posture from a supine position to a standing position, minute body motion in association with breathing during sleep, absence of body motion for a certain period of time although not sleeping (disappearance of body motion)) of the subject measured by the body motion measurement unit 32 as the additional information 54, the disease determination unit 51 can prevent occurrence of false positive and false negative of the stroke due to influence of the body motion of the subject and determine the possibility of the stroke with higher accuracy. Furthermore, in a case where the possibility of the stroke of the subject is determined further on the basis of the biological information (a blood pressure, a body temperature, a blood gas, blood glucose, etc.) of the subject measured by the biological information measurement unit 33 as the additional information 54, the disease determination unit 51 can help prevent occurrence of false positive and false negative of the stroke due to influence of the biological information of the subject and determine the possibility of the stroke with higher accuracy. Furthermore, in a case where the possibility of the stroke is determined further on the basis of the subject information (daily movement, administration of a drug/medication, a basic disease) input to the input unit 7 as the additional information 54, the disease determination unit 51 can prevent occurrence of false positive and false negative of the stroke due to influence of the daily movement, administration of a drug/medication, and the basic disease of the subject, and determine the possibility of the stroke with higher accuracy.

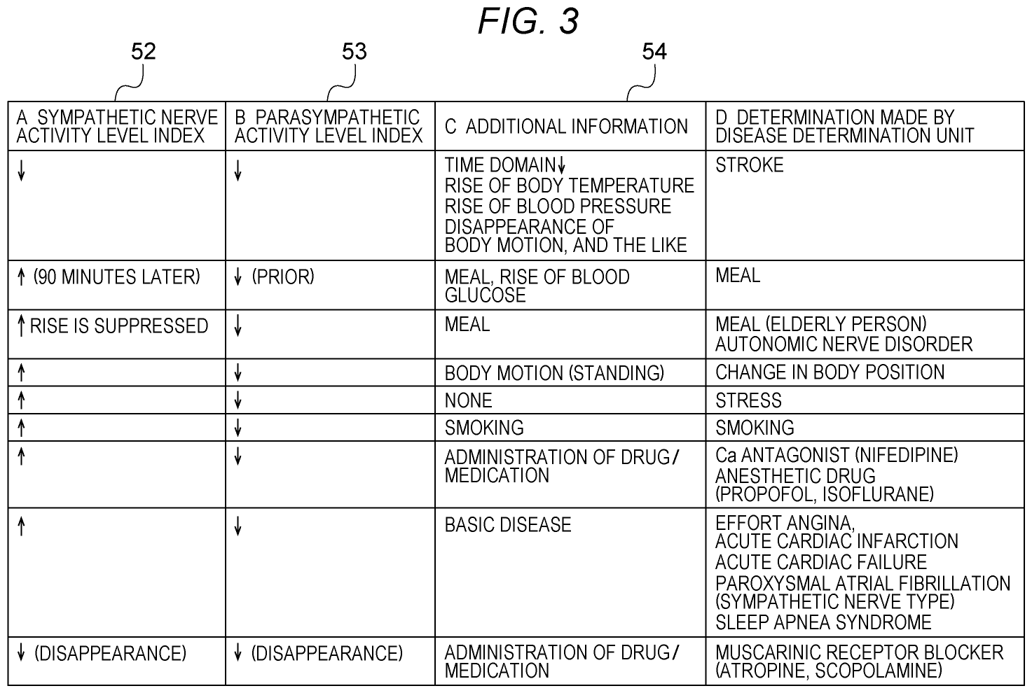

FIG. 3 is a table indicating a second specific example of determination made by the disease determination unit of the present embodiment.

As indicated in FIG. 3, in the second specific example of the determination made by the disease determination unit 51, a case where the parasympathetic nerve activity level index 53 tends to decrease will be described.

As indicated in FIG. 3, for example, if there is body motion (change in body position from lying to standing), smoking, administration/medication of a specific drug, and an onset of a specific acute disease, there is a case where the parasympathetic nerve activity level index 53 tends to decrease while the sympathetic nerve activity level index 52 tends to increase. In this case, the disease determination unit 51 determines that there is no possibility of a stroke and there is a possibility of a change in body position, smoking, or the like, as indicated in FIG. 3.

In addition, as indicated in FIG. 3, for example, if the blood glucose increases due to a meal, the parasympathetic nerve activity level index 53 decreases, and the sympathetic nerve activity level index 52 may increase after about 90 minutes have elapsed. In this case, the disease determination unit 51 determines that there is no possibility of a stroke and there is a possibility that the subject has had a meal as indicated in FIG. 3. Furthermore, as indicated in FIG. 3, for example, if an elderly person or a patient having an autonomic nerve disorder eats a meal, the parasympathetic nerve activity level index 53 tends to decrease after the meal, while rise in the sympathetic nerve activity level index 52 tends to be suppressed in some cases. In this case, the disease determination unit 51 determines that there is no possibility of a stroke, and there is a possibility that an elderly person or a patient having an autonomic nerve disorder has had a meal as indicated in FIG. 3.

On the other hand, as indicated in FIG. 3, in a case where both the sympathetic nerve activity level index 52 and the parasympathetic nerve activity level index 53 tend to decrease, the disease determination unit 51 determines that the subject has a possibility of a stroke.

According to this, an effect similar to the effect described above with reference to FIG. 2 can be obtained. Furthermore, in a case where the disease determination unit 51 determines the possibility of the stroke of the subject further on the basis of the additional information 54, an effect similar to the effect described above with reference to FIG. 2 can be obtained.

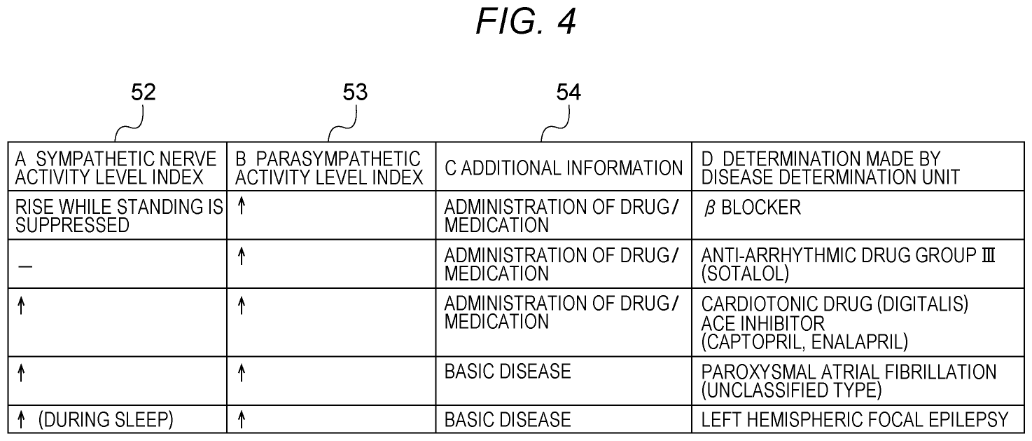

FIG. 4 is a table indicating a third specific example of determination made by the disease determination unit of the present embodiment.

As indicated in FIG. 4, in the third specific example of the determination made by the disease determination unit 51, a case where both the sympathetic nerve activity level index 52 and the parasympathetic nerve activity level index 53 tend to increase will be described.

As indicated in FIG. 4, if there is administration/medication of a specific drug and an onset of a specific acute disease, both the sympathetic nerve activity level index 52 and the parasympathetic nerve activity level index 53 may tend to increase. In this case, the disease determination unit 51 determines that there is no possibility of a stroke, and there is a possibility of administration of a drug/medication or an onset of an acute disease as indicated in FIG. 4.

On the other hand, as described above with respect to FIG. 2 and FIG. 3, in a case where both the sympathetic nerve activity level index 52 and the parasympathetic nerve activity level index 53 tend to decrease, the disease determination unit 51 determines that the subject has a possibility of a stroke.

According to this, an effect similar to the effect described above with reference to FIG. 2 can be obtained. Furthermore, in a case where the disease determination unit 51 determines the possibility of the stroke of the subject further on the basis of the additional information 54, an effect similar to the effect described above with reference to FIG. 2 can be obtained.

FIG. 5 is a table indicating a time domain index and a frequency domain index of the heart rate variability.

A numerical value of “healthy person” indicate in FIG. 5 means a numerical value when the subject is healthy and has not developed a stroke. In the example indicated in FIG. 5, the sympathetic nerve activity level index 52 (LF/HF) of a person with a stroke is 1.2 or more and 1.6 or less (i.e., 1.2 to 1.6). The sympathetic nerve activity level index 52 (LF/HF) of a healthy person is 1.5 or more and 2.0 or less (i.e., 1.5 to 2.0). Thus, in the example indicated in FIG. 5, the sympathetic nerve activity level index 52 (LF/HF) of the person with a stroke is 80% of the sympathetic nerve activity level index 52 (LF/HF) of the healthy person. As described above, if the sympathetic nerve activity level index 52 calculated by the sympathetic nerve activity level index calculation unit 42 becomes equal to or less than a first ratio with respect to the sympathetic nerve activity level index of the healthy person (that is, when the subject is healthy), the disease determination unit 51 determines that an abnormality has occurred in the sympathetic nerve activity level index 52. The first ratio can be, for example, about 80% or more and 85% or less (i.e., 80% to 85%).

In the example indicated in FIG. 5, the parasympathetic nerve activity level index 53 (HF) of the person with a stroke is 780 (ms²). The parasympathetic nerve activity level index 53 (HF) of the healthy person is 975 (ms²). Thus, in the example indicated in FIG. 5, the parasympathetic nerve activity level index 53 (HF) of the person with a stroke is 80% of the parasympathetic nerve activity level index 53 (HF) of the healthy person. As described above, if the parasympathetic nerve activity level index 53 calculated by the parasympathetic nerve activity level index calculation unit 43 becomes equal to or less than a second ratio with respect to the parasympathetic nerve activity level index of the healthy person (that is, when the subject is healthy), the disease determination unit 51 determines that an abnormality has occurred in the parasympathetic nerve activity level index 53. The second ratio can be, for example, about 80% or more and 85% or less (i.e., 80% to 85%). The second ratio may be the same as or different from the first ratio.

In the example indicated in FIG. 5, both the sympathetic nerve activity level index 52 (LF/HF) and the parasympathetic nerve activity level index 53 (HF) tend to decrease. Thus, in this case, the disease determination unit 51 determines that the subject has a possibility of a stroke.

According to this, the disease determination unit 51 determines that there is a possibility of a stroke in a case where the sympathetic nerve activity level index 52 of the subject becomes equal to or less than the first ratio with respect to the sympathetic nerve activity level index 52 of the healthy person (that is, when the subject is healthy) and the parasympathetic nerve activity level index 53 of the subject becomes equal to or less than the second ratio with respect to the parasympathetic nerve activity level index 53 of the healthy person (that is, when the subject is healthy), so that it is possible to determine the possibility of the stroke with higher accuracy in comparison with the healthy person (that is, when the subject is healthy).

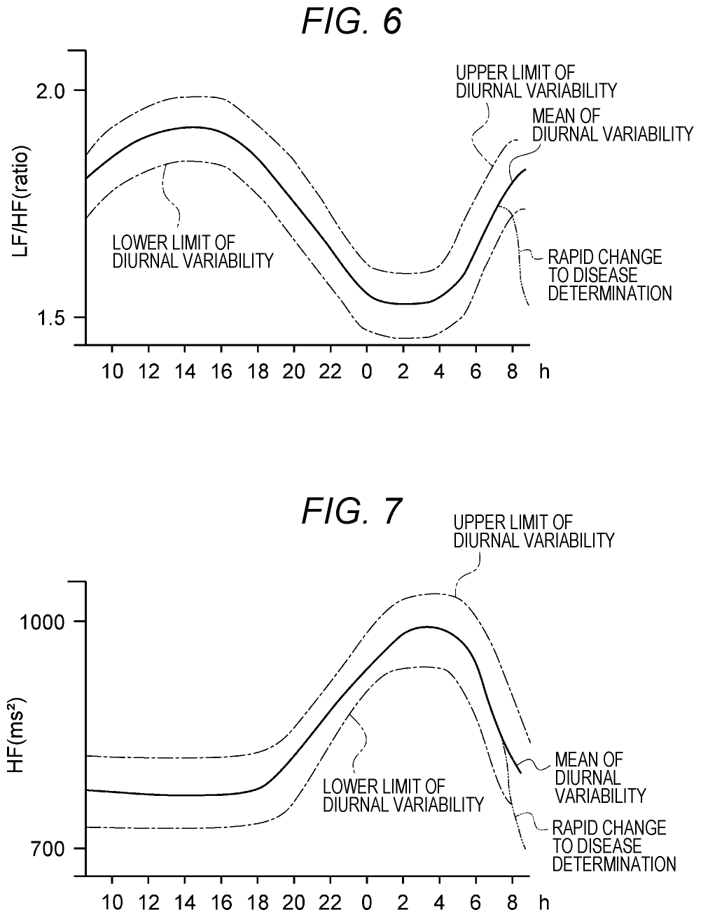

FIG. 6 is a graph indicating change tendency of the sympathetic nerve activity level index (LF/HF) at an onset of a stroke.

FIG. 7 is a graph indicating change tendency of the parasympathetic nerve activity level index (HF) at an onset of a stroke.

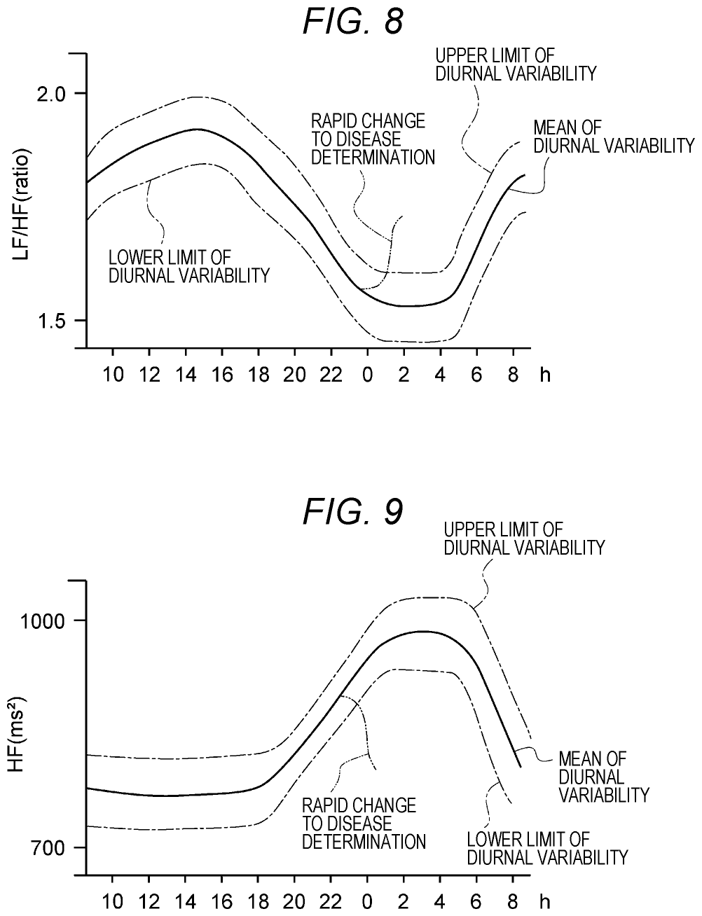

FIG. 8 is a graph indicating change tendency of the sympathetic nerve activity level index (LF/HF) at the time of a change in body position to a standing position which is one of body positions.

FIG. 9 is a graph indicating change tendency of the parasympathetic nerve activity level index (HF) at the time of a change in body position to a standing position which is one of body positions.

In the graphs indicated in FIGS. 6 to 9, two dashed-dotted curves represent a normal range of diurnal variability including variations.

First, with reference to FIGS. 8 and 9, the change tendency of the sympathetic nerve activity level index 52 (LF/HF) and the parasympathetic nerve activity level index 53 (HF) generated at the time of a change in body position to a standing position which is one of body positions will be described.

As indicated in FIG. 8, the sympathetic nerve activity level index 52 (LF/HF) at normal times tends to rise in the morning hours and decrease in the night hours. In the example indicated in FIG. 8, the sympathetic nerve activity level index 52 (LF/HF) at normal times varies within a range of about 1.5 or more and 2.0 or less (i.e., 1.5 to 2.0) in one day. In other words, the sympathetic nerve activity level index 52 (LF/HF) at normal times varies within a range of about +15% (i.e., plus or minus 15%) in one day.

Here, as indicated by a two-dot (chain curve in FIG. 8, for example, if a body position changes from a lying position to a standing position by the body motion due to desire to urinate at 0:00 in hours which are normally sleeping and lying hours, the sympathetic nerve activity level index 52 (LF/HF) rises at a ratio equal to or higher than a diurnal variability ratio (about +15%) of the sympathetic nerve activity level index 52 (LF/HF) at normal times.

As indicated in FIG. 9, the parasympathetic nerve activity level index 53 (HF) at normal times tends to decrease in the morning hours and increase in the night hours. In the example indicated in FIG. 9, the parasympathetic nerve activity level index 53 (HF) at normal times varies within a range of about 700 or more and 1000 or less (i.e., 700 to 1000) in one day. In other words, the parasympathetic nerve activity level index 53 (HF) at normal times varies within a range of about ±18% (i.e., plus or minus 18%) in one day.

Here, as indicated by a two-dot chain curve in FIG. 9, for example, if the standing position occurs at 0:00 in hours which are normally sleeping and lying hours, the parasympathetic nerve activity level index 53 (HF) decreases at a ratio equal to or higher than the diurnal variability ratio (about ±18%) of the parasympathetic nerve activity level index 53 (HF) at normal times.

In the curves indicated by two-dot chain lines in FIGS. 8 and 9, the parasympathetic nerve activity level index 53 (HF) tends to decrease, while the sympathetic nerve activity level index 52 (LF/HF) tends to increase. Thus, in this case, the disease determination unit 51 determines that there is no possibility of a stroke and there is a possibility of body motion (change in body position from a lying position to a standing position).

Next, with reference to FIGS. 6 and 7, the change tendency of the sympathetic nerve activity level index 52 (LF/HF) and the parasympathetic nerve activity level index 53 (HF) generated at an onset of a stroke will be described.

The change tendency and diurnal variability of the sympathetic nerve activity level index 52 (LF/HF) at normal times are as described above with respect to FIG. 8. The change tendency and diurnal variability of the parasympathetic nerve activity level index 53 (HF) at normal times are as described above with respect to FIG. 9. The diagnostic apparatus 2 according to the present embodiment stores the diurnal variability of the sympathetic nerve activity level index 52 (LF/HF) for one day or several days in the storage unit 8 as time-series data.

Here, as indicated by the two-dot chain curve in FIG. 6, for example, if the stroke occurs at 7:00, the sympathetic nerve activity level index 52 (LF/HF) decreases at a ratio equal to or higher than the diurnal variability ratio (about +15%) of the sympathetic nerve activity level index 52 (LF/HF) at normal times. In other words, the sympathetic nerve activity level index 52 calculated by the sympathetic nerve activity level index calculation unit 42 becomes equal to or less than a fifth ratio with respect to the sympathetic nerve activity level index 52 in the same hours on or before the previous day. The fifth ratio is a ratio equal to or higher than the diurnal variability ratio (about ±15%) of the sympathetic nerve activity level index 52 (LF/HF) at normal times, that is, for example, about 20% or more and about 25% or less (i.e., 20% to 25%) of the sympathetic nerve activity level index 52 (LF/HF) at normal times. As described above, if the sympathetic nerve activity level index 52 calculated by the sympathetic nerve activity level index calculation unit 42 becomes equal to or less than the fifth ratio with respect to the sympathetic nerve activity level index 52 in the same hours on or before the previous day, the disease determination unit 51 determines that an abnormality has occurred in the sympathetic nerve activity level index 52 (LF/HF).

In addition, as indicated in FIG. 6, in the hours around 7:00, the sympathetic nerve activity level index 52 (LF/HF) tends to rise even at normal times, as can be seen from the change in the value of the sympathetic nerve activity level index 52 (LF/HF) around 7:00 on the previous day. Thus, even if the sympathetic nerve activity level index 52 (LF/HF) rises to an extent of rising around 7:00 on the previous day in the hours around 7:00, it is not abnormal, and if this is determined to be abnormal, there is a risk of making an erroneous determination. Thus, if a threshold (first predetermined value) for abnormality determination of the sympathetic nerve activity level index 52 (LF/HF) is set on the basis of the change tendency of the sympathetic nerve activity level index 52 (LF/HF) in the same hours on or before the previous day, it is possible to prevent erroneous determination of abnormality in a case where the sympathetic nerve activity level index 52 (LF/HF) changes within a range of the diurnal variability at normal times, so that it is possible to improve accuracy of determination of the cardiovascular disease.

As indicated by the two-dot chain curve in FIG. 7, for example, if a stroke occurs at 7:00, the parasympathetic nerve activity level index 53 (HF) decreases at a ratio equal to or higher than the diurnal variability ratio (about +18%) of the parasympathetic nerve activity level index 53 (HF) at normal times. In other words, the parasympathetic nerve activity level index 53 calculated by the parasympathetic nerve activity level index calculation unit 43 becomes equal to or less than a sixth ratio with respect to the parasympathetic nerve activity level index 53 in the same hours on or before the previous day. The sixth ratio is a ratio equal to or higher than the diurnal variability ratio (about ±18%) of the parasympathetic nerve activity level index 53 (HF) at normal times, that is, for example, about 20% or more and about 25% or less (i.e., 20% to 25%) of the parasympathetic nerve activity level index 53 (HF) at normal times. The sixth ratio may be the same as or different from the fifth ratio. As described above, if the parasympathetic nerve activity level index 53 calculated by the parasympathetic nerve activity level index calculation unit 43 becomes equal to or less than the sixth ratio with respect to the parasympathetic nerve activity level index 53 in the same hours on or before the previous day, the disease determination unit 51 determines that an abnormality has occurred in the parasympathetic nerve activity level index 53 (HF).

In addition, as indicated in FIG. 7, in the hours around 7:00, the parasympathetic nerve activity level index 53 (HF) tends to decrease even at normal times, as can be seen from the change in the parasympathetic nerve activity level index 53 (HF) around 7:00 on the previous day. Thus, even if there is a decrease in the parasympathetic nerve activity level index 53 (HF) to an extent of decreasing around 7:00 on the previous day in the hours around 7:00, it is not abnormal, and if this is determined to be abnormal, there is a risk of making an erroneous determination. Thus, if a threshold (second predetermined value) for abnormality determination of the parasympathetic nerve activity level index 53 (HF) is set on the basis of the change tendency of the parasympathetic nerve activity level index 53 (HF) in the same hours on or before the previous day, it is possible to prevent erroneous determination of abnormality in a case where the parasympathetic nerve activity level index 53 (HF) changes within a range of the diurnal variability at normal times, so that it is possible to improve accuracy of determination of the cardiovascular disease.

As indicated by the two-dot chain curves in FIGS. 6 and 7, both the sympathetic nerve activity level index 52 (LF/HF) and the parasympathetic nerve activity level index 53 (HF) tend to decrease. Thus, in this case, the disease determination unit 51 determines that the subject has a possibility of a stroke.

According to this, the disease determination unit 51 determines that there is a possibility of a stroke in a case where the sympathetic nerve activity level index 52 calculated by the sympathetic nerve activity level index calculation unit 42 becomes equal to or less than the fifth ratio with respect to the sympathetic nerve activity level index 52 in the same hours on or before the previous day and the parasympathetic nerve activity level index 53 calculated by the parasympathetic nerve activity level index calculation unit 43 becomes equal to or less than the sixth ratio with respect to the parasympathetic nerve activity level index 53 in the same hours on or before the previous day. It is therefore possible to prevent occurrence of false positive and false negative of the stroke due to influence of the diurnal variability within a predetermined period of the sympathetic nerve activity level index 52 and the parasympathetic nerve activity level index 53 and to determine the possibility of the stroke with higher accuracy.

In addition, as indicated by the two-dot chain curve in FIG. 6, for example, if a stroke occurs at 7:00, the sympathetic nerve activity level index 52 (LF/HF) decreases from about 1.7 to about 1.5 within one hour from 7:00 to 8:00. In other words, the sympathetic nerve activity level index 52 (LF/HF) at 8:00 is about 88% with respect to the sympathetic nerve activity level index 52 (LF/HF) at 7:00 one hour ago. As described above, if the sympathetic nerve activity level index 52 calculated by the sympathetic nerve activity level index calculation unit 42 becomes equal to or less than the third ratio with respect to the sympathetic nerve activity level index 52 a predetermined period ago, the disease determination unit 51 determines that an abnormality has occurred in the sympathetic nerve activity level index 52.

The “predetermined period” as used herein can be, for example, about 30 minutes or more and 1 hour or less (i.e., 30 minutes to one (1) hour). The third ratio can be, for example, about 10% or more and 20% or less (i.e., 10% to 20%). In addition, instead of the ratio, for example, a value obtained by subtracting a predetermined value (for example, 0.1) from the sympathetic nerve activity level index 52 (LF/HF) one hour ago may be used as a threshold (first predetermined value) for abnormality determination. Here, the value to be subtracted from the sympathetic nerve activity level index 52 (LF/HF) one hour ago is preferably determined on the basis of the change tendency in the same hours on or before the previous day.

As indicated by the two-dot chain curve in FIG. 7, for example, if a stroke occurs at 7:00, the parasympathetic nerve activity level index 53 (HF) decreases from about 800 to about 700 within one hour from 7:00 to 8:00. In other words, the parasympathetic nerve activity level index 53 (HF) at 8:00 is about 88% of the parasympathetic nerve activity level index 53 (HF) at 7:00 one hour ago. As described above, if the parasympathetic nerve activity level index 53 calculated by the parasympathetic nerve activity level index calculation unit 43 becomes equal to or less than a fourth ratio with respect to the parasympathetic nerve activity level index 53 a predetermined period ago, the disease determination unit 51 determines that an abnormality has occurred in the parasympathetic nerve activity level index 53 (HF).

The fourth ratio can be, for example, about 10% or more and 20% or less (i.e., 10% to 20%). The fourth ratio may be the same as or different from the third ratio. In addition, instead of the ratio, as a criterion for abnormality determination, a value obtained by subtracting a predetermined value (for example, 0.5) from the value of the parasympathetic nerve activity level index 53 (HF) one hour ago is abnormal, may be used as a threshold (second predetermined value) for abnormality determination. Here, the value to be subtracted from the value of the parasympathetic nerve activity level index 53 (HF) one hour ago is preferably determined on the basis of the change tendency of the parasympathetic nerve activity level index 53 (HF) on or before the previous day.

As indicated by the two-dot chain curves in FIGS. 6 and 7, both the sympathetic nerve activity level index 52 (LF/HF) and the parasympathetic nerve activity level index 53 (HF) tend to decrease. Thus, in this case, the disease determination unit 51 determines that the subject has a possibility of a stroke.

According to this, the disease determination unit 51 determines that there is a possibility of a stroke in a case where the sympathetic nerve activity level index 52 calculated by the sympathetic nerve activity level index calculation unit 42 becomes equal to or less than the third ratio with respect to the sympathetic nerve activity level index 52 a predetermined period ago and the parasympathetic nerve activity level index 53 calculated by the parasympathetic nerve activity level index calculation unit 43 becomes equal to or less than the fourth ratio with respect to the parasympathetic nerve activity level index 53 the predetermined period ago. It is therefore possible to prevent occurrence of false positive and false negative of the stroke due to influence of the diurnal variability of the sympathetic nerve activity level index 52 and the parasympathetic nerve activity level index 53 and to determine the possibility of the stroke with higher accuracy.

In addition, the disease determination unit 51 may determine that there is a possibility of a stroke in a case where the sympathetic nerve activity level index 52 calculated by the sympathetic nerve activity level index calculation unit 42 becomes equal to or less than a first predetermined value smaller than the sympathetic nerve activity level index 52 a predetermined period ago and the parasympathetic nerve activity level index 53 calculated by the parasympathetic nerve activity level index calculation unit 43 becomes equal to or less than a second predetermined value smaller than the parasympathetic nerve activity level index 53 the predetermined period ago. The “predetermined period” as used herein can be, for example, about 30 minutes or more and 1 hour or less (i.e., 30 minutes to one (1) hour).

The first predetermined value is determined on the basis of a change in the sympathetic nerve activity level index 52 of a predetermined period ago and a change in the sympathetic nerve activity level index 52 in the same hours on or before the previous day. In addition, the second predetermined value is determined on the basis of a change in the parasympathetic nerve activity level index 53 a predetermined period ago and a change in the parasympathetic nerve activity level index 53 in the same hours on or before the previous day.

According to this, the disease determination unit 51 can prevent occurrence of false positive and false negative of the stroke due to influence of the diurnal variability of the sympathetic nerve activity level index 52 and the parasympathetic nerve activity level index 53, and can determine the possibility of the stroke with higher accuracy.

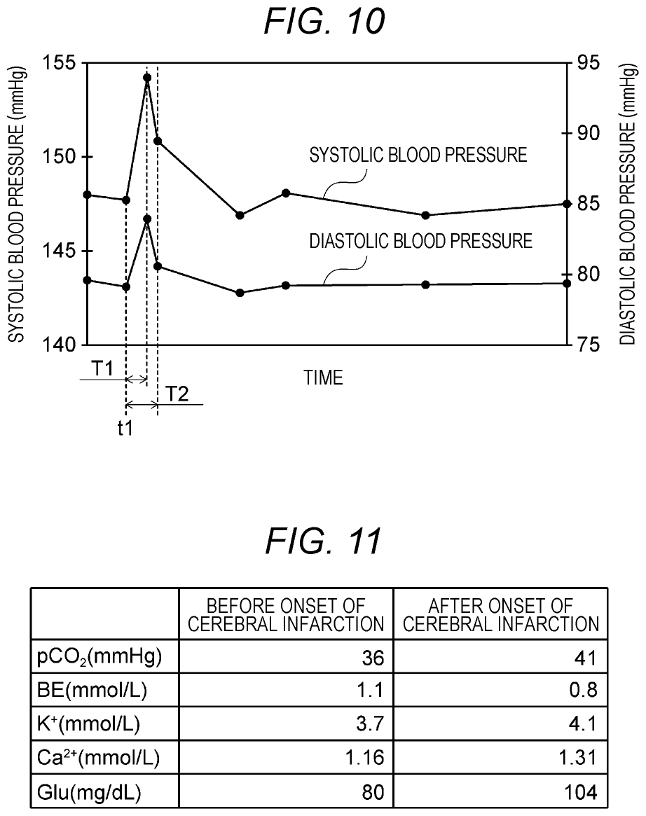

FIG. 10 is a graph for explaining a case where the disease determination unit of the present embodiment determines the possibility of the stroke further on the basis of a blood pressure as the additional information.

As indicated in FIG. 10, if a stroke occurs at time t1, the blood pressure rises at a predetermined ratio or higher with respect to the blood pressure at rest within a predetermined period T1. The “predetermined period T1” as used here can be, for example, about 5 minutes or more and 10 minutes or less (i.e., 5 minutes to 10 minutes). The “predetermined ratio” as used here can be, for example, about 5%.

In the example indicated in FIG. 10, a systolic blood pressure at rest is about 147 mmHg. The systolic blood pressure at an onset of a stroke is about 155 mmHg. In other words, the systolic blood pressure at the time of an onset of a stroke is increased by about 5% with respect to the systolic blood pressure at rest. A diastolic blood pressure at rest is about 80 mmHg. The diastolic blood pressure at the time of an onset of a stroke is about 84 mmHg. In other words, the diastolic blood pressure at the time of an onset of a stroke is increased by 5% with respect to the diastolic blood pressure at rest.

Then, if a stroke occurs, a state in which the blood pressure is higher than the blood pressure at rest continues for a predetermined period T2 or more. The “predetermined period T2” as used here can be, for example, about 30 minutes or more and 60 minutes or less (i.e., 30 minutes to 60 minutes).

The disease determination unit 51 determines the possibility of the stroke of the subject not only on the basis of the change tendency of the sympathetic nerve activity level index 52 and the parasympathetic nerve activity level index 53 but also on the basis of the change tendency of the blood pressure of the subject. This enables the disease determination unit 51 to further prevent occurrence of false positive and false negative of the stroke and determine the possibility of the stroke with higher accuracy.

FIG. 11 is a table for explaining a case where the disease determination unit of the present embodiment determines the possibility of the stroke further on the basis of a blood gas as the additional information.

If a stroke occurs, the blood gas varies at a predetermined ratio or higher within a predetermined period. The “predetermined period” as used here can be, for example, about 5 minutes or more and 10 minutes or less (i.e., 5 minutes to 10 minutes). The “predetermined ratio” as used here can be, for example, about 5%.

Items of the blood gas test available for detecting an onset of cerebral infarction include hydrogen ion concentration (pH), a carbon dioxide partial pressure (pCO2), an oxygen partial pressure (pO2), base excess (BE), bicarbonate ion concentration (HCO3−), sodium ion concentration (Na+), potassium ion concentration (K+), calcium ion concentration (Ca2+), a blood glucose level (Glu), and the like. FIG. 11 is an example of blood gas changes before an onset of cerebral infarction and immediately after the onset of cerebral infarction. In the example indicated in FIG. 11, the measured values of pCO2, K+, Ca2+, and Glu are increased, and the measured value of BE is decreased after the onset of cerebral infarction relative to before the onset of cerebral infarction. The change ratio of each measured value is about 10% or more and 30% or less (i.e., 10% to 30%).

The disease determination unit 51 determines the possibility of the stroke of the subject not only on the basis of the change tendency of the sympathetic nerve activity level index 52 and the parasympathetic nerve activity level index 53 but also on the basis of the change tendency of the blood gas of the subject. This enables the disease determination unit 51 to further prevent occurrence of false positive and false negative of the stroke and determine the possibility of the stroke with higher accuracy.

Note that the disease determination unit 51 may determine the possibility of the stroke on the basis of not only the blood pressure described above with reference to FIG. 10 and the blood gas described above with reference to FIG. 11 but also a body temperature as the additional information. In this case, for example, if the body temperature of the subject increases from normal to 37.5 degrees or higher within 5 minutes, the disease determination unit 51 determines that an abnormality has occurred in the body temperature of the subject. In other words, the possibility of the stroke of the subject is determined not only on the basis of the change tendency of the sympathetic nerve activity level index 52 and the parasympathetic nerve activity level index 53 but also on the basis of the change tendency of the body temperature of the subject. This enables the disease determination unit 51 to further prevent occurrence of false positive and false negative of the stroke and determine the possibility of the stroke with higher accuracy.

As described above, according to the diagnostic apparatus 2 of the present embodiment, the disease determination unit 51 determines the possibility of the cardiovascular disease of the subject on the basis of the change tendency of the sympathetic nerve activity level index 52 and the change tendency of the parasympathetic nerve activity level index 53, so that it is possible to prevent occurrence of false positive and false negative of the cardiovascular disease and determine the possibility of the stroke with higher accuracy. For example, the disease determination unit 51 determines that there is a possibility of an onset of a stroke in a case where both the sympathetic nerve activity level index 52 calculated by the sympathetic nerve activity level index calculation unit 42 and the parasympathetic nerve activity level index 53 calculated by the parasympathetic nerve activity level index calculation unit 43 tend to decrease. Thus, the disease determination unit 51 can further prevent occurrence of false positive and false negative of the cardiovascular disease and determine the possibility of the cardiovascular disease with higher accuracy.

The sympathetic nerve activity level index calculation unit 42 calculates the sympathetic nerve activity level index 52 on the basis of the heart rate variability to be used as the autonomic nerve index. The parasympathetic nerve activity level index calculation unit 43 calculates the parasympathetic nerve activity level index 53 on the basis of the heart rate variability to be used as the autonomic nerve index. For example, in a case where the heartbeat interval calculation unit 41 calculates the heartbeat interval on the basis of the electrocardiogram and the invasive blood pressure data, the disease determination unit 51 can determine the possibility of the cardiovascular disease with higher accuracy. For example, in a case where the heartbeat interval calculation unit 41 calculates the heartbeat interval on the basis of data measured by a wearable terminal, or the like, it is possible to prevent a scale of the diagnostic apparatus 2 from becoming relatively large and to reduce a burden associated with measurement to be felt by the subject, and the disease determination unit 51 can more easily determine the possibility of the cardiovascular disease even in a state where the subject is living his/her daily life.

In addition, in a case where the disease determination unit 51 determines the possibility of the cardiovascular disease on the basis of the change tendency (time domain analysis, or the like) of the heartbeat interval calculated by the heartbeat interval calculation unit 41, the possibility of the cardiovascular disease can be determined with higher accuracy.

In addition, the sympathetic nerve activity level index calculation unit 42 calculates the sympathetic nerve activity level index 52 by analyzing a frequency component of the heart rate variability using a frequency component analysis method such as fast Fourier transform, for example. In addition, the parasympathetic nerve activity level index calculation unit 43 calculates the parasympathetic nerve activity level index 53 by analyzing a frequency component of the heart rate variability using a frequency component analysis method such as fast Fourier transform, for example. Thus, the disease determination unit 51 can determine the possibility of the cardiovascular disease with higher accuracy.

The detailed description above describes to a diagnostic apparatus and a diagnostic program for determining a possibility of a cardiovascular disease such as a stroke. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A diagnostic apparatus comprising:
a sympathetic nerve activity level index calculation unit configured to calculate a sympathetic nerve activity level index indicating a sympathetic nerve activity level;
a parasympathetic nerve activity level index calculation unit configured to calculate a parasympathetic nerve activity level index indicating a parasympathetic nerve activity level; and
a disease determination unit configured to determine a possibility of a cardiovascular disease of a subject on a basis of change tendency of the sympathetic nerve activity level index calculated by the sympathetic nerve activity level index calculation unit and change tendency of the parasympathetic nerve activity level index calculated by the parasympathetic nerve activity level index calculation unit.

2. The diagnostic apparatus according to claim 1, wherein the disease determination unit is configured to determine that there is the possibility of the cardiovascular disease in a case where both the sympathetic nerve activity level index calculated by the sympathetic nerve activity level index calculation unit and the parasympathetic nerve activity level index calculated by the parasympathetic nerve activity level index calculation unit tend to decrease.

3. The diagnostic apparatus according to claim 1, wherein the disease determination unit is configured to determine that there is the possibility of the cardiovascular disease in a case where the sympathetic nerve activity level index calculated by the sympathetic nerve activity level index calculation unit becomes equal to or less than a first ratio with respect to the sympathetic nerve activity level index of the subject when the subject is healthy, and the parasympathetic nerve activity level index calculated by the parasympathetic nerve activity level index calculation unit becomes equal to or less than a second ratio with respect to the parasympathetic nerve activity level index of the subject when the subject is healthy.

4. The diagnostic apparatus according to claim 1, wherein the disease determination unit is configured to determine that there is the possibility of the cardiovascular disease in a case where the sympathetic nerve activity level index calculated by the sympathetic nerve activity level index calculation unit becomes equal to or less than a third ratio with respect to the sympathetic nerve activity level index a predetermined period ago, and the parasympathetic nerve activity level index calculated by the parasympathetic nerve activity level index calculation unit becomes equal to or less than a fourth ratio with respect to the parasympathetic nerve activity level index the predetermined period ago.

5. The diagnostic apparatus according to claim 1, wherein the disease determination unit is configured to determine that there is the possibility of the cardiovascular disease in a case where the sympathetic nerve activity level index calculated by the sympathetic nerve activity level index calculation unit becomes equal to or less than a first predetermined value smaller than the sympathetic nerve activity level index a predetermined period ago, and the parasympathetic nerve activity level index calculated by the parasympathetic nerve activity level index calculation unit becomes equal to or less than a second predetermined value smaller than the parasympathetic nerve activity level index the predetermined period ago.

6. The diagnostic apparatus according to claim 5, wherein the first predetermined value is determined on a basis of a change in the sympathetic nerve activity level index the predetermined period ago and a change in the sympathetic nerve activity level index in the same hours on or before the previous day, and/or the second predetermined value is determined on a basis of a change in the parasympathetic nerve activity level index the predetermined period ago and a change in the parasympathetic nerve activity level index in the same hours on or before the previous day.

7. The diagnostic apparatus according to claim 3, wherein the cardiovascular disease is a stroke.

8. The diagnostic apparatus according to claim 1, wherein the disease determination unit is configured to determine that there is the possibility of the cardiovascular disease in a case where the sympathetic nerve activity level index calculated by the sympathetic nerve activity level index calculation unit becomes equal to or less than a fifth ratio with respect to the sympathetic nerve activity level index in the same hours on or before the previous day, and the parasympathetic nerve activity level index calculated by the parasympathetic nerve activity level index calculation unit becomes equal to or less than a sixth ratio with respect to the parasympathetic nerve activity level index in the same hours.

9. The diagnostic apparatus according to claim 1, further comprising:
a heartbeat interval calculation unit configured to calculate a heartbeat interval of the subject; and
wherein the sympathetic nerve activity level index calculation unit is configured to calculate the sympathetic nerve activity level index on a basis of heart rate variability indicating variability of the heartbeat interval calculated by the heartbeat interval calculation unit, and the parasympathetic nerve activity level index calculation unit is configured to calculate the parasympathetic nerve activity level index on a basis of the heart rate variability.

10. The diagnostic apparatus according to claim 9, wherein the disease determination unit is configured to determine the possibility of the cardiovascular disease further on a basis of change tendency of the heartbeat interval calculated by the heartbeat interval calculation unit.

11. The diagnostic apparatus according to claim 9, wherein
the sympathetic nerve activity level index calculation unit is configured to calculate the sympathetic nerve activity level index on a basis of frequency component analysis of the heart rate variability, and
the parasympathetic nerve activity level index calculation unit is configured to calculate the parasympathetic nerve activity level index on a basis of the frequency component analysis of the heart rate variability.

12. The diagnostic apparatus according to claim 1, further comprising:
a biological information measurement unit configured to measure biological information including one or more of a blood pressure, a body temperature, or a blood gas of the subject; and
wherein the disease determination unit is configured to determine the possibility of the cardiovascular disease further on a basis of change tendency of the biological information measured by the biological information measurement unit.

13. The diagnostic apparatus according to claim 1, further comprising:
a subject information input unit to which subject information including one or more of information regarding daily movement of the subject, information regarding a drug to be used by the subject, or information regarding a basic disease of the subject is input; and
wherein the disease determination unit is configured to determine the possibility of the cardiovascular disease further on a basis of the subject information input to the subject information input unit.

14. The diagnostic apparatus according to claim 1, further comprising:
a body motion measurement unit configured to measure body motion of the subject; and
wherein the disease determination unit is configured to determine the possibility of the cardiovascular disease further on a basis of the body motion measured by the body motion measurement unit.

15. The diagnostic apparatus according to claim 1, further comprising:
a blood glucose measurement unit configured to measure blood glucose of the subject; and
wherein the disease determination unit is configured to determine the possibility of the cardiovascular disease further on a basis of change tendency of the blood glucose measured by the blood glucose measurement unit.

16. A non-transitory computer-readable medium storing a diagnostic program, which causes a computer to execute:
a sympathetic nerve activity level index calculation procedure of calculating a sympathetic nerve activity level index indicating a sympathetic nerve activity level;
a parasympathetic nerve activity level index calculation procedure of calculating a parasympathetic nerve activity level index indicating a parasympathetic nerve activity level; and
a disease determination procedure of determining a possibility of a cardiovascular disease of a subject on a basis of change tendency of the sympathetic nerve activity level index calculated by the sympathetic nerve activity level index calculation procedure and change tendency of the parasympathetic nerve activity level index calculated by the parasympathetic nerve activity level index calculation procedure.

17. A method for determining a possibility of a cardiovascular disease of a subject comprising:
calculating a sympathetic nerve activity level index indicating a sympathetic nerve activity level;
calculating a parasympathetic nerve activity level index indicating a parasympathetic nerve activity level; and
determining the possibility of the cardiovascular disease of the subject on a basis of change tendency of the sympathetic nerve activity level index calculated by the sympathetic nerve activity level index calculation unit and change tendency of the parasympathetic nerve activity level index calculated by the parasympathetic nerve activity level index calculation unit.

18. The method according to claim 17, further comprising:
determining that there is the possibility of the cardiovascular disease in a case where both the sympathetic nerve activity level index calculated by the sympathetic nerve activity level index calculation unit and the parasympathetic nerve activity level index calculated by the parasympathetic nerve activity level index calculation unit tend to decrease.

19. The method according to claim 17, further comprising:
determining that there is the possibility of the cardiovascular disease in a case where the sympathetic nerve activity level index calculated by the sympathetic nerve activity level index calculation unit becomes equal to or less than a first ratio with respect to the sympathetic nerve activity level index of the subject when the subject is healthy, and the parasympathetic nerve activity level index calculated by the parasympathetic nerve activity level index calculation unit becomes equal to or less than a second ratio with respect to the parasympathetic nerve activity level index of the subject when the subject is healthy.

20. The method according to claim 17, further comprising:
determining that there is the possibility of the cardiovascular disease in a case where the sympathetic nerve activity level index calculated by the sympathetic nerve activity level index calculation unit becomes equal to or less than a third ratio with respect to the sympathetic nerve activity level index a predetermined period ago, and the parasympathetic nerve activity level index calculated by the parasympathetic nerve activity level index calculation unit becomes equal to or less than a fourth ratio with respect to the parasympathetic nerve activity level index the predetermined period ago.
